(19) United States
(12) Patent Application Publication
(10) Pub. No.: US 2017/0135806 A1
(43) Pub. Date: May 18, 2017

(54) DEVICE FOR REPAIRING ANEURYSM WITHIN MULTI-BRANCH VESSEL

(71) Applicant: Mark P. Ombrellaro, Bellevue, WA (US)

(72) Inventor: Mark P. Ombrellaro, Bellevue, WA (US)

(21) Appl. No.: 15/353,712

(22) Filed: Nov. 16, 2016

Related U.S. Application Data
(60) Provisional application No. 62/255,908, filed on Nov. 16, 2015.

(51) Int. Cl. A61F 2/07; A61F 2/856

(52) U.S. Cl. CPC A61F 2/07; A61F 2/856; A61F 2250/0064; A61F 2002/061

(57) ABSTRACT

A stent graft system includes a plurality of stents that are configured to be assembled in situ to provide an aortal stent graft assembly. The system may include a tubular segment that is configured to engage healthy aortic tissue, a plurality of segments configured to sealingly engage the tubular segment and comprising one or a plurality of vents or side channels that are oriented proximally and are configured to receive and seal with smaller-diameter stent grafts. In some embodiments, the system further includes a tubular segment having a bifurcated distal end. For example, the system may be assembled to provide a flow channel and support structure for stent grafts that engage one or more of the right and left iliac arteries, the celiac artery, the superior mesenteric artery, and the renal arteries.

### CROSS-REFERENCE TO RELATED APPLICATION

This application claims the benefit of U.S. Provisional Application No. 62/255,908, filed Nov. 16, 2015; the entire disclosure of said application is hereby incorporated by reference.

### BACKGROUND

Endovascular repair of aneurysms of the aorta, first described by Parodi in 1992, have become a mainstay of treatment for this condition since devices approved by the U.S. Food and Drug Administration for endovascular repair of aneurysms were released in 1999. The concept is to reline the weakened section of the aorta by placing a prosthetic aortic replacement (or endograft) within the damaged segment of the artery itself. Visually, this amounts to installing a new pipe or channel (graft) inside the confines of a damaged pipe (the aneurysmal segment of artery) to relieve pressure in the damaged pipe to keep it from rupturing. The endovascular graft is secured to non-dilated (or more normal) arterial tissue both above and below the dilated aneurysmal segment, which effectively excludes blood flow and arterial pressure from reaching the weakened segment of the aortic wall. These devices have been shown to reduce the risk of death from aortic rupture comparable to a standard open arterial aneurysm repair, but with significantly less risk, less blood loss, a shorter hospital stay, and a quicker overall return of patients to active function.

The primary requirements for an endograft to effectively exclude the damaged segment of arterial wall are fixation and seal. “Fixation” means that the device is securely held in position to minimize the chance of migration or slippage over time and under the force of arterial blood pressure. “Seal” means creating a fluid-impervious barrier zone where the device is in contact with the “normal” segments of artery both above and below the treated aneurysmal segment. An effective seal prevents leakage of blood between the device and the aortic wall which would otherwise result in undesirable pressurization of the aneurysmal segment.

The aorta, the main arterial pipeline leaving the heart, is generally cane-shaped and is conventionally described in several anatomical sections, as briefly described below, along with a summary of major arteries that branch from the aorta.

The section of the aorta attached to, and extending upwardly from, the left ventricle of the heart is referred to as the ascending aorta. The coronary arteries originate from the ascending aorta.

The arcuate section extending from the ascending aorta is referred to as the transverse arch. The arteries of the arms, head, and neck (for standard anatomy, the three branches from proximal to distal are the innominate or brachiocephalic artery, the left common carotid artery, and the left subclavian artery) engage the transverse arch.

The section extending downwardly into the chest from the transverse arch is the descending thoracic aorta. The section extending downwardly from the diaphragm and into the abdomen is referred to as the visceral segment of the abdominal aorta, followed by the infrarenal segment of the abdominal aorta. The infrarenal segment of the abdominal aorta terminates by splitting into the right and left iliac arteries, which serve as the perfusion pathway for the pelvis and lower extremities. The visceral segment of the abdominal aorta provides blood to the celiac artery and to the superior mesenteric artery (SMA), which provides the primary perfusion to the stomach, intestines, and other organs. The visceral segment of the abdominal aorta also provides blood to the renal arteries, which provide perfusion to the kidneys.

Aortic endografting was originally described to treat aneurysms of the infrarenal segment of the abdominal aorta. The endograft was essentially a straight tube or “Y”-shaped configuration that allowed treatment of the aortic segment from below the renal arteries to either a more distal location in the aorta itself or the iliac arteries. Commercially available devices also became available in larger sized tubes to treat aneurysm of the descending thoracic aorta, from the limit of the left subclavian artery to the celiac artery. Standard infrarenal devices are also limited in application by anatomic morphology of the abdominal aortic aneurysm with respect to the infrarenal landing (seal) zones. Recent guidelines of the minimum requirements for standard commercially available endografts are a neck length of 10 mm, neck diameter of <32 mm, and angulation of <60-90 degrees. Approximately 25%-75% of all abdominal aortic aneurysms remain unsuitable for standard endovascular aortic aneurysm repair.

The challenges that remain are the ability to treat aneurysmal segments of the aorta from which the various branch vessels arise, i.e., the transverse arch and the visceral abdominal aortic segments. The primary challenge of endovascular aneurysm repair of these aortic segments is the ability to maintain perfusion to the branch vessels when the required seal zones span the branch vessel. With current devices, sealing a stent graft in the more normal segment of upstream artery would result in occluding flow through the downstream branches.

A prior art branch vessel graft method and system to address this problem is disclosed in U.S. Patent Application Publication No. 2008/0167704, to Wright et al., which is hereby incorporated by reference. The endografts disclosed in Wright et al. require custom stent grafts with fenestrations positioned for a particular application, and the insertion and sealing of branch connections at right angles to the main stent graft. Right-angle branch grafts present difficulties in installation because the surgeon must navigate the guiding device, e.g., guide wire to and through the fenestration.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates application of an endograft system in accordance with the present invention in an aortic aneurysm in the visceral segment of the abdominal aorta;

FIG. 2 illustrates schematically an exemplary endograft component engaging conventional stent grafts in accordance with the present invention;

FIG. 3 illustrates schematically another embodiment of an endograft component in accordance with the present invention;

FIGS. 4A-4D illustrate schematically four basic configurations for endograft components suitable for use in the major combinations of branch vessels in Zones 6-8 of the aortic system in accordance with the present invention; and

FIG. 5 illustrates schematically a configuration for an endograft component in accordance with the present invention and suitable for use in the aortic transverse arch.

### DETAILED DESCRIPTION

Currently, to treat aneurysms involving aortic segments with major branches, custom made devices are manufactured that may include scallops (cutaways), fenestrations (holes), branches (attached graft arms), and/or diameter-reducing ties based on the specific anatomy of the individual patient. These custom-made devices are time-consuming to make, extremely expensive, technically challenging to place and properly reconstruct, and require significantly longer radiation and contrast exposure to achieve placement. They are also associated with a higher morbidity and mortality rate when compared to standard thoracic or infrarenal abdominal aortic aneurysm repair. Placement of these devices also frequently requires additional arterial access points, typically through the left subclavian or carotid artery, in order to place the branch grafts themselves.

The Society for Vascular Surgery has proposed an endovascular classification system for abdominal aortic aneurysms based on the proximal sealing zones of aortic endografts. These zones are determined by the location of the seal zone necessary to treat the aneurysm and the visceral vessels that would be covered in the process and requiring reconstruction. Zone 8 aneurysms, for example, typically require a graft to seal with the renal arteries. Conventionally, this would require two fenestrations (one per renal artery), additional fenestrations for major accessory renal arteries, and a scallop to allow perfusion to the SMA. Zone 7 aneurysms typically require the graft to cover and seal the SMA and renal arteries. This would require three fenestrations (one per main renal and one for the SMA), additional fenestrations for major accessory renal arteries, and a scallop to allow perfusion to the celiac artery. Zone 6 aneurysms typically require the graft to cover and seal the celiac artery, the SMA, and the renal arteries. This would require four fenestrations (one per main renal, one for the SMA, and one for the celiac artery), additional fenestrations for major accessory renal arteries, or quadruple branch grafts, one for each major branch.

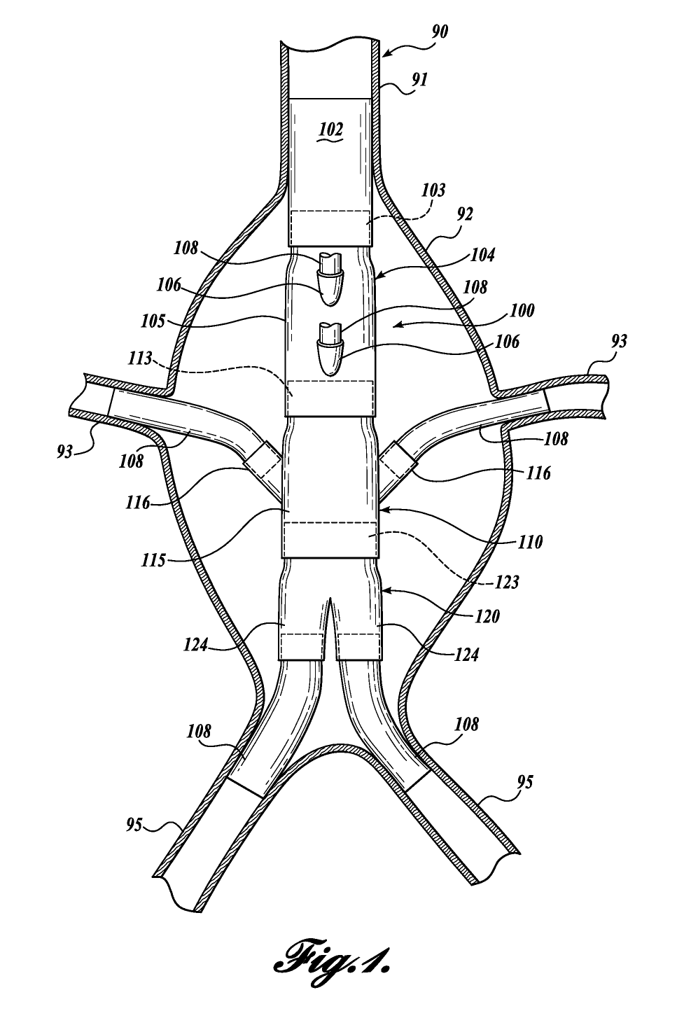

FIG. 1 illustrates a stent graft assembly 100 for an abdominal aortic aneurysm in an aorta 90 in accordance with the present invention. The assembly 100 includes a proximal segment 102 comprising a tubular stent graft having a first end that engages healthy aortic tissue 91 above the aneurysm and a second end that extends distally into the enlarged aneurysmal channel.

A second segment 104 comprises a tubular portion 105 having a proximal end 103 that extends into and sealingly engages the proximal segment 102. One or more vents or side channels 106 (two shown) are configured to engage and seal with smaller diameter stent grafts 108. The stent grafts 108 may be configured to sealingly engage and provide perfusion to the superior mesenteric artery and/or the celiac artery (not shown), for example.

In an alternative assembly, for example, in situations wherein the aneurysm is less extensive in the proximal direction, the proximal end 103 of the second segment 104 may extend directly into the healthy aortic tissue 91 to sealingly engage with the aorta 90, eliminating the need for the proximal segment 102.

A third segment 110 comprises a tubular portion 115 having a proximal end 113 that extends into and sealingly engages with the distal end of the second segment 104. The third segment 110 includes one or more vents or side channels 116 that are configured to engage and seal with smaller-diameter stent grafts 108. For example, the stent grafts 108 may be configured to sealingly engage and provide perfusion to the renal arteries 93.

An optional fourth segment 120 is shown in the assembly 100, comprising a bifurcated segment having a larger-diameter proximal end 123 that extends into and sealingly engages the distal end of the third segment 110. The fourth segment 120 includes a bifurcated distal end defining two flow channels 124 that may directly engage the common iliac arteries 95, or may be configured to sealingly retain tubular stent grafts 108 that engage the common iliac arteries 95. It will be apparent to persons of skill in the art that the fourth segment 120 would not be required if there is sufficient non-aneurysmal aortic tissue between the renal arteries 93 and the common iliac arteries 95 to provide a seal at the distal end of the segment 110.

As shown in FIG. 1, in the current embodiment at least some of the components in the assembly 100 are short, modified tubular structures, for example, segments 104 and 110. The segments 104, 110 may be in a standard range of diameters and lengths typical for aortic endografts. Stents used to treat aortic aneurysms and sealably join with existing stent grafts currently have diameters in the range of 12-46 mm. Lengths of 30-60 mm are currently believed to be suitable. Although the currently preferred diameter and length are described, it is clear that the components can assume any appropriate diameter or length.

It is contemplated that the segments, e.g., segments 102, 104, 110, 120, may be provided in kit form, optionally in a plurality of standard size ranges, for example, as discussed above. With a kit, the surgeon can select the required components for a particular situation.

The individual components can be made of polyethylene terephthalate PET (e.g., Dacron®), polytetrafluoroethylene (PTFE), or any other material suitable for blood vessel replacement. Additionally, the external aspect of the fabric layer is supported with an exoskeleton to provide outward expansile radial force to the components, as are known in the art. The exoskeleton can be constructed of any type of metal material used for arterial stenting, most commonly nickel titanium alloy (also known as nitinol).

In an alternative embodiment, a fluid-tight fabric channel or bladder is provided that can be filled with a fluid or polymer to act as a support system. Additionally, attachment hooks, as are known in the art, are placed along the body of the device to provide a mechanism for active fixation with the non-aneurysmal aortic wall tissue and with other endograft components.

Sealing aortic stent grafts is typically achieved by oversizing the device components by 10%-20% with respect to the upstream and downstream arterial wall or endograft components in order to produce a fluid-impervious friction seal between the device and its point of attachment. The typical overlapping segment or seal zone is ideally a minimum of 10 mm between the device and the non-aneurysmal arterial wall and at least 20 mm between device components. In an alternative embodiment, a fluid-tight fabric channel or bladder that can be filled with a fluid or polymer is provided and configured to act as a sealing gasket between individual components in the endograft assembly 100, and between the components and the atrial wall.

Radio-opaque markers are also incorporated into one or more of the components serving as identifiers for all major aspects of the device, such as the proximal and distal ends of the device itself, and the location of all retrograde perfusion channels, as discussed below.

The current system includes a fixed combination of angulated vents or side perfusion channels, oriented to have the side channels opening proximally and the base of each side channel towards the distal downstream end of the device. The side channel may be configured, for example, to form an angle of 45 degrees to the body of the device. It will be readily apparent that other angles are possible, although it is believed that optimally the side channels will be at an angle of less than 90 degrees with respect to the central axis of the device. The length of the side channel may be varied to accommodate particular applications. For example, the side channel sections in some embodiments may be 5-10 mm in some embodiments, with a diameter ranging from 3-10 mm.

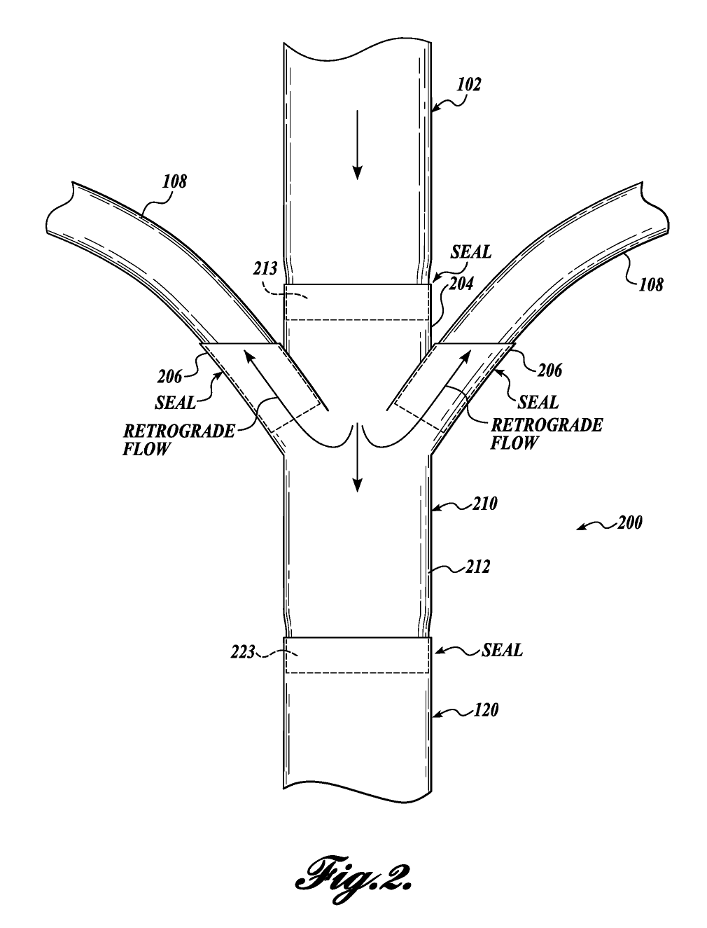

As shown in FIGS. 2 and 3, the side channels can be formed, for example, by adding an additional fabric material sleeve (and skeleton) extending outwardly from the cylindrical component body, creating an external side channel that is in fluid communication with the inside of the main flow channel; or by creating a slit in the tubular body material itself and sewing or sealing a seam (or pleat) of an appropriate length, directed inward into the main body of the device to create an internal side channel. The seam would allow for the creation of a 5-10 mm long side channel or vented tunnel within the main body of the device. The side channel is configured to establish a retrograde perfusion between the device and any branch vessel by linking the side channel, for example, with a standard covered stent via femoral access.

The orientation of the side channels 106, 116 allows for the main flow channel of the segment body to be accessed and entered from below via standard femoral arterial access, and exited through the side channel 106, 116 in what is referred to as a retrograde direction (against the normal direction of blood flow). Passing through the side channel 106, 116 in this direction allows shaped catheters or steerable sheaths to easily traverse the wall of the device from inside out, and into the origin of a target branch vessel. After the catheter or sheath is in proper position, a guide wire can be directed into the specific target branch vessel and used as a rail for the delivery and deployment of a standard, covered stent device to create a covered channel connecting the segment 104, 110 with the target branch artery.

FIG. 2 illustrates another assembly 200 with an embodiment of a segment 210 in accordance with the present invention. The segment 210 comprises an upper tubular proximal portion 204 that engages a proximal stent graft 102 to form a proximal seal 213, a pair of vents or side channels 206 that sealingly engage smaller-diameter stent grafts 108, and a lower tubular portion 212 that engages distal stent graft 120 to form a distal seal 223. The side channels 206 and stent grafts 108 are configured to produce a local retrograde blood flow. This retrograde configuration facilitates installation of the assembly from the distal direction.

In another embodiment, shown in FIG. 3, a segment 310 is configured with side channels 306 formed within the main body 304 of the segment 310. The smaller-diameter stent grafts 108 are retained and seal with the respective side channel 306. The side channels 306 are configured to produce a retrograde flow. An upper seal 307 in the segment 310 prevents blood outflow above the side channels 306.

It will be appreciated that segments may be configured with side channels at any point along the body of the device, and that a single device may have more than one side channel. When multiple side channels are incorporated into the device, they may be stacked in a line or offset with respect to one another. Offsets may be aligned (e.g., vents all pointing straight up but in different lanes), angulated (e.g., stacked in a line but the opening of the side channel directed at different angles), or a combination of both. It is important to provide sufficient room between side channels to accommodate placement of a covered stent device to create a covered channel connecting the device and the target branch artery while not interfering with other side channels in the area.

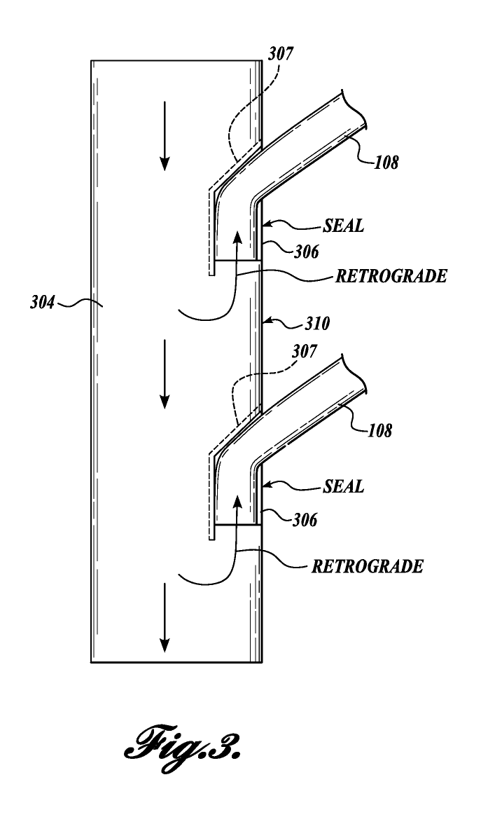

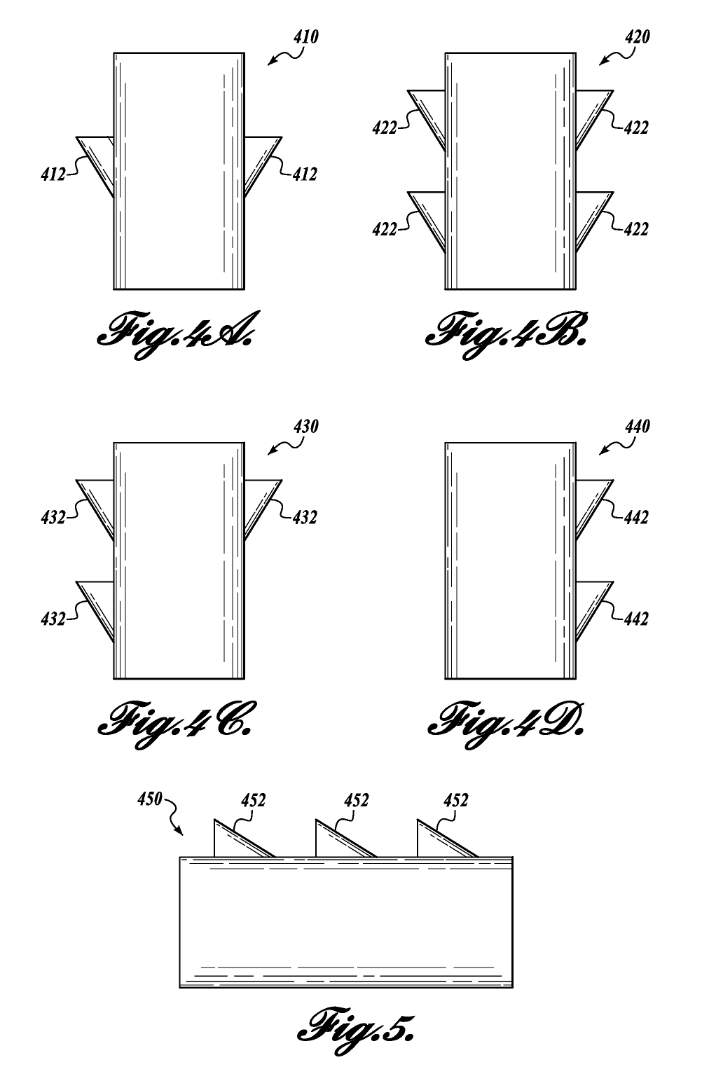

When considering the most common anatomical configurations encountered during the repair of Zone 6-8 regions of the visceral abdominal aorta, four primary component designs would allow for the treatment of all of the major anatomic variations in an “off-the-shelf” manner, as shown in FIGS. 4A-4D. A fifth design, shown in FIG. 5, would allow for treatment of branch artery configurations found in the transverse arch of the thoracic aorta.

In FIG. 1 the assembly may be assembled essentially from the proximal end to the distal end. The proximal segment 102, if required, is installed first, in healthy aortic tissue above the SMA and celiac arteries. The second segment 104 may then be inserted partially into the distal end of the proximal segment 102 and expanded to form the seal 103. Similarly, the third segment 110 (if required) may be inserted partially into the distal end of the second segment 104 and expanded to form the seal 113. Then the fourth segment 120 (if required) is inserted partially into the distal end of the third segment 110 to form the seal 123. The stent grafts 108 may then be inserted and sealed accordingly.

As illustrated in FIG. 2, the assembly may alternatively be assembled in the distal to proximal direction. For example, the segment 120 may be placed initially, extending proximally. The segment 210 may then be inserted through the segment 120, such that a distal end overlaps in the seal 223, and expanded to form the seal. A more proximal segment 102 may then be inserted through the segment 210 and expanded to form the seal 213.

When describing these five primary component designs, the following convention is used: If the device is viewed looking top down though the main flow channel, zero degrees is at the 12 o'clock position and oriented in situ to the patient's back (posterior). The front of the device is at 180 degrees or the 6 o'clock position and oriented in situ to the patient's front (anterior). The 90-degree position is at the 3 o'clock position and oriented in situ to the patient's left, and 270 degrees is at the 9 o'clock position and oriented in situ to the patient's right.

Configuration 1 (FIG. 4A): The device or segment 410 includes two side channels 412 located at the same level with openings 180 degrees apart. Typically the side channels 412 are located at 90 degrees and 270 degrees. This allows for treatment of aneurysms requiring a seal at Zone 8 with preservation of flow to two branch vessels opposite each other, such as the right and left renal arteries, with single renal artery anatomy.

Configuration 2 (FIG. 4B): The device or segment 420 includes four side channels 422, two side channels each stacked in a linear series with the stacks being 180 degrees apart; and the side channel openings of each stack located at the same level relative to each other. Typically the stacks would be located at 90 degrees and 270 degrees. This allows for treatment of aneurysms requiring a seal at Zone 8 with preservation of flow to four branch vessels, two pairs located on opposite sides of the aorta, such as duplicate right and left renal arteries (double renal artery per side anatomy).

Configuration 3 (FIG. 4C): The device or segment 430 includes three side channels 432, two side channels each linearly aligned on one side and a single side channel 432 located at the same level as the upper most side channel in the stack, 180 degrees apart. Typically the stack would be located at 90 degrees and the single side channel at 270 degrees. This allows for treatment of aneurysms requiring a seal at Zone 8 with preservation of flow to three branch vessels (one pair and a single branch located on opposite sides of the aorta), such as a single renal artery on one side and duplicate renal arteries on the other. With this configuration, one device can be used in a duplicate right or left situation by rotating the device 180 degrees to line up with the appropriate channels.

Configuration 4 (FIG. 4D): The device or segment 440 includes two side channels 422 aligned in series. Typically the stack would be located at 180 degrees. This would be for treatment of aneurysms requiring a seal at Zones 6 and 7 with preservation of flow to two branch vessels in a line, one below the other, such as the celiac and the SMA.

Configuration 5 (FIG. 5): The device or segment 450 includes three vents or side channels 452 in a linear series. The vents or side channels 452 in this embodiment are positioned to provide access to the innominate or brachiocephalic artery, the left common carotid artery, and the left subclavian artery, for example. This allows for treatment of aneurysms requiring a seal in the ascending aorta with preservation of flow to three branch vessels in a line, one below the other, such as the innominate, left carotid, and left subclavian arteries. The device can be rotated for proper alignment of the side channels with the branch vessel origins.

A novel system of modular components is disclosed herein, which can be delivered via standard femoral or iliac arterial access. The vents or side channels do not have to line up exactly with the target branch arteries to be connected, which allows for more flexibility in treating aneurysms and to adapting to the specific anatomical features of the particular patient. The system also provides greater technical flexibility during aneurysm repairs, providing more repair options, and allowing the surgeon to work in more favorable areas of the dilated aorta and still get the desired seals with the target branch vessels. It also provides for the use of off-the-shelf components rather than requiring individual custom components. The vents or side channels also allow for better seals with the branch vessel stent grafts because the side channels provide an internal sleeve. The inventor has found that when trying to stent or perform additional treatments from within an aneurysm, the large dilated aneurysm can be a hindrance. The large space does not provide any back support from the opposite side of the arterial wall for the sheaths and catheters because the opposite wall is too far away. When the stiffer devices are advanced over the guiding wire and resistance is met from either a bend in the artery or narrowing (stenosis or blockage), the forces work to effectively push the wire and the system away from the target artery and out of the branch. The narrow vents, as well as working inside the segment itself (with a smaller-diameter than the aneurysm itself), provide additional back support to counter resistance, facilitating the placement of the components.

In a current embodiment, the system comprises a plurality of stent graft components that may be used alone or in combination with existing infrarenal or thoracic aortic endograft systems to allow effective treatment of branch vessel segments of aneurysmal aorta. It is contemplated that each component type may be provided in a range of standard diameters and lengths consistent with existing aortic endografts. For example, some of the components in the system may be used as upward extensions for infrarenal aortic endografts, distal extensions for thoracic endografts, or central connecting pieces linking thoracic to infrarenal endografts to allow for effective treatment of aortic aneurysms involving the visceral vessels.

While illustrative embodiments have been illustrated and described, it will be appreciated that various changes can be made therein without departing from the spirit and scope of the invention.

The embodiments of the invention in which an exclusive property or privilege is claimed are defined as follows:

1. A stent graft kit comprising a plurality of stents that are configured to be assembled to provide an aortal stent graft assembly, wherein the stent kit comprises:
a first stent having a proximal end and a distal end, wherein the proximal end is configured to be inserted into a channel defined by an aorta and to sealingly engage healthy aortic tissue;
a second stent having a proximal end configured to be inserted into and sealingly engage the distal end of the first stent, a distal end, and at least one side channel disposed between the proximal end and the distal end of the second stent, wherein the at least one side channel has an external opening that opens in the proximal direction; and
at least one elongate stent graft having a proximal end that is configured to be inserted into the external opening and to sealingly engage the at least one side channel of the second stent and a distal end that is configured to be inserted into and sealingly engage another artery.

2. The stent graft kit of claim 1, further comprising:
a third stent having a proximal end configured to sealingly engage the distal end of the second stent, a distal end, and at least two side channels disposed between the proximal end and the distal end of the third stent, wherein the at least two side channels each have an external opening that opens in the proximal direction.

3. The stent graft kit of claim 2, further comprising:
a fourth stent having a proximal end configured to sealingly engage the distal end of the second stent and a distal end defining two bifurcated channels.
